(19) United States
(12) Patent Application Publication
(10) Pub. No.: US 2025/0222197 A1
(43) Pub. Date: Jul. 10, 2025

(54) Surgical Irrigation Clip

(71) Applicant: MAKO Surgical Corp., Weston, FL (US)

(72) Inventors: Andrew Jacob Nelson, New York City, NY (US); Shane Carey, Carrignavar (IE)

(73) Assignee: MAKO Surgical Corp., Weston, FL (US)

(21) Appl. No.: 19/014,319

(22) Filed: Jan. 9, 2025

Related U.S. Application Data
(60) Provisional application No. 63/619,342, filed on Jan. 10, 2024.

(51) Int. Cl. A61M 3/02; B05B 15/62; F16L 3/00

(52) U.S. Cl. CPC A61M 3/0279; B05B 15/62; F16L 3/00; A61M 2207/00

(57) ABSTRACT

A connector for attaching a fluid source to a surgical tool includes a body extending along an axis and defining a lumen within the body. The lumen has a lumen proximal end configured to receive fluid from the fluid source. The fluid is transported through the body by the lumen. An attachment portion extends from the body transverse to the axis and is configured to releasably attach the body to an exterior surface of the surgical tool. The body and the attachment portion are integrally formed by additive manufacturing.

### CROSS-REFERENCE TO RELATED APPLICATIONS

The subject application claims priority to and all benefits of U.S. Provisional Patent App. No. 63/619,342, filed Jan. 10, 2024, the entire contents of which are hereby incorporated by reference.

### BACKGROUND

Conventional medical procedures routinely involve the use of surgical tools to assist surgical professionals in approaching, viewing, manipulating, or otherwise effecting treatment at localized surgical sites. Certain applications, such as those which involve the use of high-speed drills, rotating burs, open-window shavers, and the like, necessarily result in the accumulation of heat and debris at the surgical site. Often, surgical systems employ irrigation systems which include a fluid source connected to a surgical tool via a flexible tube. The surgical tool typically employs one or more clips or fasteners to facilitate attachment over the irrigation tube to the surgical tool for concurrent movement.

Conventional irrigation clips have several shortcomings. For example, many clips require a technician to manually feed the flexible tube through the clip. This process can be a cumbersome and adds additional setup time in the operating room. Moreover, this process can result in improper installation or damage to the tubing. Further, conventional irrigation clips are not single use, or disposable, and must be sterilized for further use. However, considering the small internal structures of such clips, the sterilization process may not be adequate to ensure proper sterility. Additionally, conventional irrigation clips do not afford flexibility in how the fluid is dispersed. Different surgical procedures, workflows, or surgeon preferences may demand the fluid be dispersed in numerous ways. Yet, conventional irrigation clips simply cannot meet these demands. Furthermore, conventional clips are often formed of metal or steel. The extra weight (even if a few ounces) can potentially affect the performance of the surgical tool to which the clip is attached.

As such, there remains a need in the art for a surgical irrigation apparatus which may overcome at least some of the disadvantages mentioned above, which can be used in connection with different types of surgical tools used in a broad array of medical and surgical procedures, and which strikes a substantial balance between usability, functionality, and manufacturing cost while, at the same time, affording consistent and reliable irrigation in use.

### SUMMARY

This Summary introduces a selection of concepts in a simplified form that are further described below in the Detailed Description below. This Summary is not intended to limit the scope of the claimed subject matter nor identify key features or essential features of the claimed subject matter.

According to a first aspect, a connector for attaching a fluid source to a surgical tool is provided. The connector includes a body extending along an axis and defining a lumen within the body. The lumen has a lumen proximal end configured to receive fluid from the fluid source. The fluid is transported through the body by the lumen. An attachment portion extends from the body transverse to the axis and is configured to releasably attach the body to an exterior surface of the surgical tool. The body and the attachment portion are integrally formed by additive manufacturing.

According to a second aspect, a method is provided of manufacturing a connector for attaching a fluid source to a surgical tool, the method includes: forming, by additive manufacturing, a body extending along an axis and defining a lumen within the body, and wherein the lumen is formed with a lumen proximal end configured to receive fluid from the fluid source, and wherein the lumen is formed to transport the fluid through the body; and forming, by additive manufacturing, an attachment portion extending from the body transverse to the axis and wherein the attachment portion is formed to releasably attach the body to an exterior surface of the surgical tool.

According to a first aspect, a connector for attaching a fluid source to a surgical tool is provided. The connector includes a body extending along an axis and defining a lumen within the body. The lumen has a lumen proximal end configured to receive fluid from the fluid source. The fluid is transported through the body by the lumen. The body integrally defines a cavity opposite the lumen proximal end. A nozzle is configured to be disposed in the cavity. An attachment portion extends from the body transverse to the axis and is configured to releasably attach the body to the surgical tool. The body and the attachment portion are integrally formed by additive manufacturing.

Any of the above aspects can be combined in part, or in whole, with any other aspect. Any of the aspects above can be combined in part, or in whole, with any of the following implementations.

The connector can be formed of, or comprise, a resin-based material, a polymer or a polymeric material, or thermoset plastic. The connector can be formed by DLP or SLA additive manufacturing.

The body can define a cavity opposite the lumen proximal end. The cavity can receive a nozzle that fluidly couples with the lumen. The nozzle can include a first end and a second end opposite the first end. The nozzle includes at least one nozzle orifice. The nozzle orifice extends through the nozzle from the first end to the second end. The nozzle orifice has an inlet at the second end for receiving fluid from the lumen and an outlet for expelling fluid. The body and the nozzle can be cured together such that the nozzle is sealed to the body, and more specifically, with a water-tight seal. Anti-rotational features can be formed on the nozzle and/or the cavity to prevent rotation of the nozzle within the cavity. The nozzle can include a metal or a metal alloy, or a resin-based material or polymeric material.

Alternatively, the body can define at least orifice at the lumen distal end. The orifice can have a cross-sectional area smaller than the cross-sectional area of the lumen at the lumen proximal end. The at least one orifice formed (in the nozzle or body), can vary with respect to one or more of: cross-sectional area, angle of extension relative to the axis of the body, and/or spray angle.

The lumen can have a shaft segment extending from the lumen proximal end towards the orifice. A cross-sectional area of the shaft segment can be variable or uniform. The lumen can have a transitional hollow disposed between the shaft segment and the orifice. The transitional hollow can have a cross-sectional area that decreases as the transitional hollow extends from the shaft segment towards the orifice. The transitional hollow can have a tapered, conical or funnel configuration. The body can have a retention feature configured to receive a tube that is coupled to the fluid source and configured to fluidly couple the lumen with the fluid source. The retention feature can annularly extend about an exterior surface of the body near the lumen proximal end. The lumen can be formed such that the fluid source tube does not enter the lumen.

The connector can include an indicium formed on any part of the connector. The indicium can be formed on the attachment portion. The indicium can indicate the direction of fluid flow through the connector. The connector can include projection(s) to assist with gripping the connector. The connector can be formed with texture that serves a purpose. The attachment portion can be a pair of opposing arms, such as arc-shaped arms, or L-shaped arms. The attachment portion arms can be flexible and movable between a disengaged position and an engaged position. The attachment portion can be a closed geometry, for example, through which the instrument is inserted. The body can include a mechanism configured to rotate to adjust a spray pattern of the fluid.

Any of the above aspects can be combined in full or in part. Any features of the above aspects can be combined in full or in part. Any of the above implementations for any aspect can be combined with any other aspect. Any of the above implementations can be combined with any other implementation whether for the same aspect or a different aspect.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a perspective view of an example surgical system whereby a connector is attached to a surgical tool and connected to a fluid source, according to one example.

FIG. 2 is a perspective view of the connector attached to the surgical tool, where a tube is attached to the connector and the connector is dispensing fluid, according to one example.

FIG. 3 is a perspective view of the connector, according to one implementation, wherein the attachment portion includes a part of arc shaped arms and an indicium (e.g., arrow) to indicate fluid flow direction.

FIG. 4 is a cross-sectional perspective view of the connector, according to one implementation.

FIG. 5 is a cross-sectional view of an implementation of the connector where the connector has more than one orifice.

FIG. 6 is a perspective view of an implementation of the connector where the connector includes a nozzle with a tube attached.

FIG. 7 is a cross-sectional exploded view of the implementation of FIG. 6 taken along the sectional line 7, with the nozzle separated from the connector and with the tube attached.

FIG. 8 is a cross-sectional view of the implementation of FIG. 6 taken along the sectional line 7, with the nozzle engaged in the cavity of the connector.

FIGS. 9A-9D show multiple implementations of orifice configurations.

FIG. 10 is another implementation of the connector where a cavity within the connector and the nozzle include corresponding anti-rotational features.

FIG. 11 is another implementation of the connector where a feature within the connector limits the depth of the nozzle when the nozzle is inserted from a connector distal end.

FIG. 12 is another implementation of the connector whereby the nozzle is insertable through the connector proximal end.

FIG. 13 is another implementation of the connector with the connector including an adjuster configured to adjust a spray pattern of fluid expelled from the connector.

FIG. 14 is a sectional view of the implementation of FIG. 13 taken along sectional line 14.

FIG. 15 is another implementation of the connector including the attachment portion with L-shaped arms.

### DETAILED DESCRIPTION

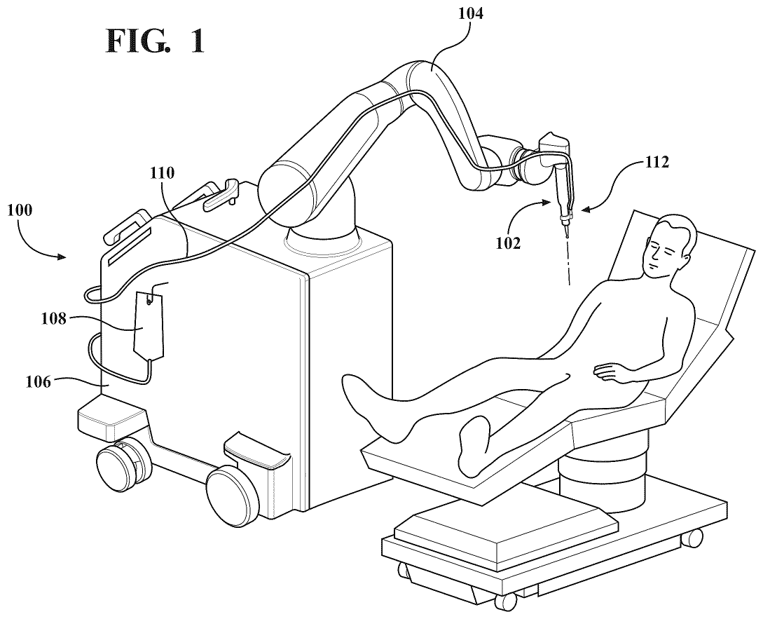

Referring to the Figures, wherein like numerals indicate like parts throughout the several views, a robotic surgical system 100 is shown in FIG. 1. In FIG. 1, a connector 112 is releasably attached to a surgical tool 102. The surgical tool 102 can be coupled to a robot 104, which is coupled to a robot cart 106. A tube 110 connects to a connector proximal end 114 of the connector 112 to a pump (not shown) behind the robot cart 106. Another tube 110 connects the pump to a fluid source 108. The fluid source 108 is attached to the robot cart 106 but may be placed elsewhere, including on a mobile apparatus. The connector 112 can be utilized for any type of surgical procedure, surgical system, or surgical tool 102 other than that shown in FIG. 1. For example, the connector 112 can be attached to a hand-held surgical tool 102 and the surgical tool 102 can be any type of surgical tool 102, such as a cutting bur, a saw, a reamer, a drill, a driver, an ultrasonic aspirator, an arthroscope, and the like. The connector 112 can be used to provide fluid for any type of surgical procedure, such as partial or total knee arthroplasty, hip arthroplasty, shoulder arthroplasty, spinal procedures, or the like.

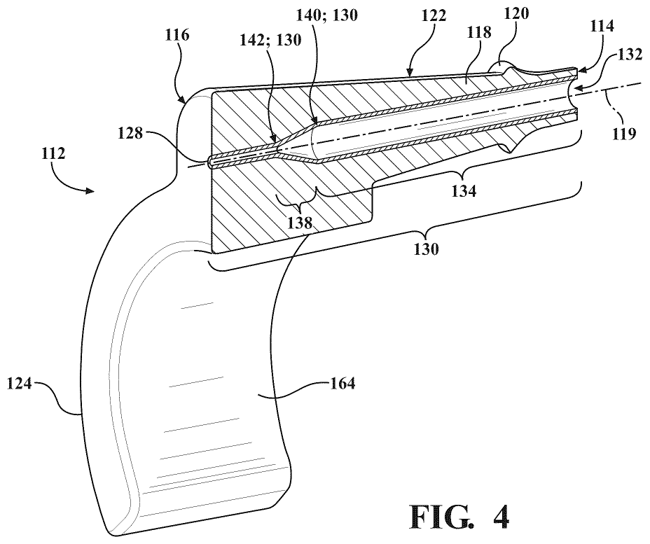

The connector 112 attaches the fluid source 108 to the surgical tool 102. As shown in FIG. 4, the connector 112 includes a body 118 extending along an axis 119 and defining a lumen 130 within the body 118. The body 118 is formed to include a void that defines the lumen 130. The lumen 130 has a lumen proximal end 132 configured to receive fluid from the fluid source 108. The fluid is transported through the body 118 via the lumen 130 and through a lumen distal end opposite the lumen proximal end 132. The lumen distal end may be located at the connector distal end 116. Advantageously, the fluid is transported directly through the lumen 130 formed in the body 118 of the connector 112. The lumen 130 assists in providing internal pressure for expelling the fluid from the connector 112. In turn, this design eliminates the need to manually feed the irrigation source tube through the lumen 130. An attachment portion 124 extends from the body 118 transverse to the axis 119. The attachment portion 124 is configured to releasably attach the body 118 to an exterior surface of the surgical tool 102.

The body 1118 and the attachment portion 124 of the connector 112 may be formed by additive manufacturing and can comprise a resin-based material, a polymeric material, a photopolymer material, or thermoset plastic. Additive manufacturing can include Digital Light Processing (DLP), 3D printing, stereolithography, selective laser sintering, fused deposition modeling (FDM), droplet jetting technologies, high area rapid printing (HARP), ultraviolent light activated resin printers, and the like. For example, additive manufacturing may include forming the connector 112 with a resin-based material, layer by layer, with a DLP machine that uses a projected (e.g., UV or Arc Lamp) light source to cure each layer, one at a time. The resin-based material then becomes a polymeric material. Alternatively, when SLA printing is utilized, the SLA machine can use a laser that traces out a path with the laser, curing along that path. Other types of additive manufacturing techniques are contemplated.

The additive manufacturing process may include with using a digital representation of a 3D model of the connector 112. 3D printing software may take the digital representation of the 3D connector 112 and cut the computer model into a series of cross-sectional layers. The cross-sectional layers of the computer model may be generated using additive manufacturing software. A computing device can execute the additive manufacturing software which is referred to as a “slicer” or “slicing software.” The slicing software can store the cross-sectional layers in the computing device as data referred to as a “slicer file” or “g-code.” The slicer file can provide instructions to the 3D printer to execute a specific order of linear movements. The 3D printer includes an extruder or fixation laser that completes the series of cross-sectional layers. As the extruder or fixation laser moves to the specific locations, a precise amount of material is dispensed to complete the cross-sectional layer. The cross-sectional layers can be formed upon the previous layer vertically, and the layers bind together momentarily after the material is dispensed. This process is repeated until the connector 112 is completed.

It is advantageous to have the connector 112 formed by additive manufacturing as the process allows overcoming limitations presented by conventional connectors. Unlike conventional connectors, the connector 112 formed by additive manufacturing enables user-defined or customized specifications of the connector 112. This customization may be useful as different surgical procedures, workflows, or surgeon preferences may demand the fluid be dispersed in numerous ways. Moreover, utilizing additive manufacturing enables the production of the connector 112 with intricate design features discussed below. Further, the connector 112 is a robust delivery of fluid in a disposable or individual use configuration, thereby reducing cost and eliminating the need for sterilization. Lastly, the additive manufacturing process is adaptable and expeditious, which allows for design changes to be quickly implemented in a production environment.

Referring to the lumen 130, and as illustrated in FIG. 4, in some implementations, the lumen 130 defines at least one orifice 128. The at least one orifice 128 is located at or near the lumen distal end or connector distal end 116. The at least one orifice 128 may be a hollow (such as a cylindrical hollow) that starts within the body 118 and terminates at (or forms) the lumen distal end. In such cases, the at least one orifice 128 may have any suitable length, such as 1 mm, 3 mm. etc. In other examples, the orifice 128 may be an opening that is located or formed at the lumen distal end (e.g., without extending into the body). This orifice 128 may be integrally formed into the body 118, e.g., by the additive manufacturing process.

The orifice 128 has a cross-sectional area that is smaller than the cross-sectional area of the lumen 130 at the lumen proximal end 132. For the purposes of this disclosure, any reference to “cross-sectional area” is the area of the cross-section taken perpendicular to the axis 119. The orifice 128 has a smaller cross-sectional area than the cross-sectional area of the lumen proximal end 132 to increase the pressure of the fluid before the fluid leaves the lumen 130. The lumen 130 creates a pathway for the fluid to travel from the connector proximal end 114 to the connector distal end 116. The lumen 130 may have a (hollow) shaft segment 134 extending from the lumen proximal end 132 towards the orifice 128. The cross-sectional area of the shaft segment 134 may be variable or uniform. In some implementations, the shaft segment 134 is a cylindrical hollow. The orifice 128 may have a smaller cross-sectional area than the cross-sectional area of the shaft segment 134.

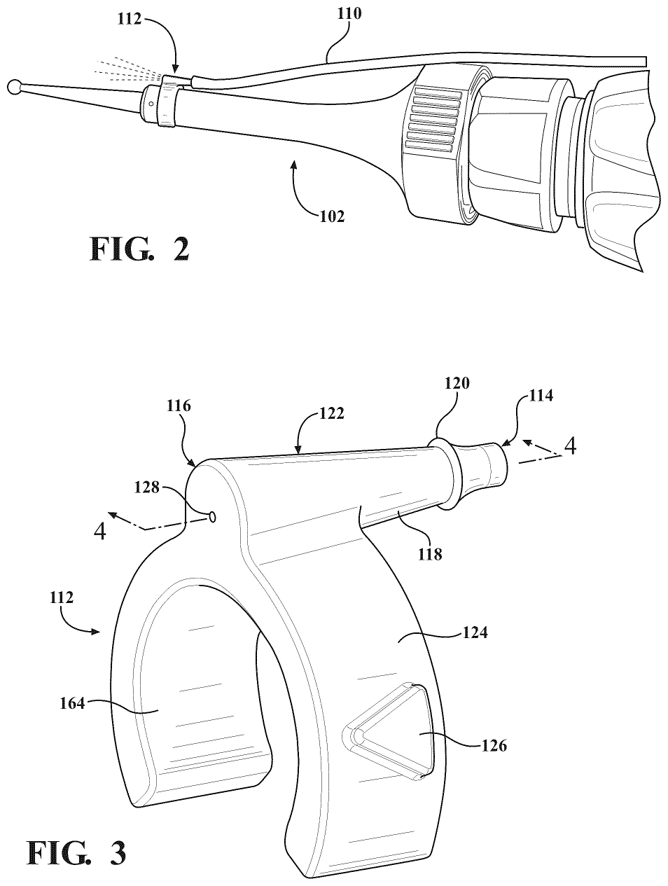

Referring still to FIG. 4, in some implementations, the lumen 130 may have a transitional hollow 138 disposed between the shaft segment 134 and the orifice 128. The transitional hollow 138 can have a conical or funnel configuration, for example. The transitional hollow 138 configuration can include any type of tapered design besides a funnel or cone. An advantage of the transitional hollow 138 is the prevention of blockage at the orifice 128 and providing a smooth transitioning of fluid pressure. The transitional hollow 138 has a cross-sectional area that decreases as the transitional hollow 138 extends from the shaft segment 134 towards the orifice 128. The transitional hollow 138 includes a transitional hollow proximal end 140 and a transitional hollow distal end 142. The transitional hollow proximal end 140 is adjacent to the shaft segment 134 and they share the same cross-sectional area as shown in FIG. 4. The transitional hollow distal end 142 is adjacent to the inlet 148 of the orifice 128 and they share the same cross-sectional area, also shown in FIG. 4. When the connector 112 is in operation, fluid travels through the shaft segment 134 to the transitional hollow 138 and the fluid is expelled through the orifice 128 of the lumen 130. Fluid being expelled through the orifice 128 of the lumen 130 is shown in FIG. 2.

As shown throughout FIGS. 2-8 and 10-15, in some implementations, the body 118 includes a retention feature 120. The retention feature 120 can annularly extends about an exterior surface 122 of the body 118 near the lumen proximal end 132. The retention feature 120 is configured to receive the tube 110 coupled to the fluid source 108. The retention feature 120 is configured to engage and secure the tube 110 to the connector 112 and fluidly couple the lumen 130 with the tube 110. The body 118 is shaped conically, where the outer diameter of the connector proximal end 114 is smaller than the outer diameter of the connector distal end 116. The retention feature 120 may be a rigid part, formed by additive manufacturing, which extends outward, from the exterior surface 122 of the body 118. In some cases, the retention feature 120 may be a separate component that wraps around the body 118. As best shown in FIG. 6, as the tube 110 is pushed over the retention feature 120, the tube 110 expands, creating an interference fit. The interference fit between the tube 110 and the retention feature 120 prevents the tube 110 from easily detaching from the connector 112. The retention feature 120 could have other configurations that advantageously enable connection of the tube 110 to the exterior of the body 118 without requiring the tube 110 to be manually inserted through the connector 112. The body 118 can have an arm portion defined between the attachment portion 124 and the retention feature 120. The arm portion can have a conical or frustoconical shape and can have a diameter that gradually decreases in size as the arm portion extends from the attachment portion to the retention feature 120.

Referring to the attachment portion 124, as shown throughout FIGS. 1-8 and 10-15, the attachment portion 124 is flexible due to the material by which it is produced from additive manufacturing. The attachment portion 124 can attach to any one of a plurality of surgical tools 102. In some implementations, the attachment portion 124 is located towards the connector distal end 116 of the body 118. In one implementation, as shown in FIG. 6, the attachment portion 124 is further defined as a pair of opposing arms. The pair of opposing arms are flexible and movable between a disengaged position and an engaged position. The disengaged position is shown in FIG. 3 as the connector 112 is shown disengaged from the surgical tool 102. In the disengaged position, the pair of opposing arms are in a relaxed state. In contrast, the attachment portion 124 is shown in the engaged position in FIG. 2. In the engaged position, the pair of opposing arms expand further outward to accommodate the geometry of the surgical tool 102 and engage the surgical tool 102 in a pressure-fit relationship such that the geometry of the surgical tool 102 prevents the attachment portion 124 from returning to the disengaged position. The opposing arms can be c-shaped, as shown in FIG. 3, for example. C-shaped arms are suitable for attaching to a round surgical tool 102, such as a nose tube of a rotary cutting tool. Alternatively, depending on the application, the opposing arms may form other shapes to conform to the respective surgical tool 102. For example, in FIG. 15, the arms each have an L-shape. Furthermore, it is contemplated that the attachment portion 124 may form a closed geometry, like a closed “O” shape or a closed rectangular shape.

In some implementations, the attachment portion 124 is coupled to the body 118 at an angle which helps to align the angle of the fluid flow with the surgical tool 102. The attachment portion 124 includes an inner surface 164 that sits on the surgical tool 102. The inner surface 164 may include a range of surface texture and design to accommodate the type of surgical tool 102. The inner surface 164 may be smooth to allow easy movement of the connector 112 along an axis of the surgical tool 102 or rough to prevent movement. The inner surface 164 can include a range of extrusions or pockets to conform to the surface of the surgical tool 102.

In some implementations, as best shown in FIGS. 3, 6, 13, and 15, the connector 112 may include a visual indicator or indicium 126 to convey information to the user. The indicium 126 may be formed by additive manufacturing and may be thereby integrated into the connector 112. In the examples shown, the indicium 126 is provided on the attachment portion 124. Additionally, or alternatively, the indicium 126 can be provided on any portion of the body 118. As shown, the indicium 126 can be a projection that protrudes from the exterior surface of the connector 112. In other examples, the indicium 126 can be flush with the exterior surface of the connector 112 or embossed or recessed below the exterior surface of the connector 112. In the illustrated examples, the indicium 126 is a directional indicator (e.g., an arrow) that conveys the direction of fluid flow. In other words, the directional indicator informs the user that the connector 112 should be installed onto the surgical instrument such that the directional arrow points towards the surgical site. Other indicium 126 can convey installation instructions, a part number, or the like. The indicium 126 can include graphic representations or imagery. When the indicium 126 is formed as a projection, the indicium can serve a dual purpose of assisting the user with gripping the connector 112. In other examples, one or multiple projections can be formed on the attachment portion 124 solely to assist with gripping without providing any type of indicia. Additionally, the additive manufacturing techniques described herein enable the connector 112 to be formed with any suitable texture on the exterior surface. The texture can have one or many purposes, such as to assist with gripping the connector, to visually distinguish the connector (e.g., from the environment), the provide anti-reflective properties, to provide water-wicking, or the like.

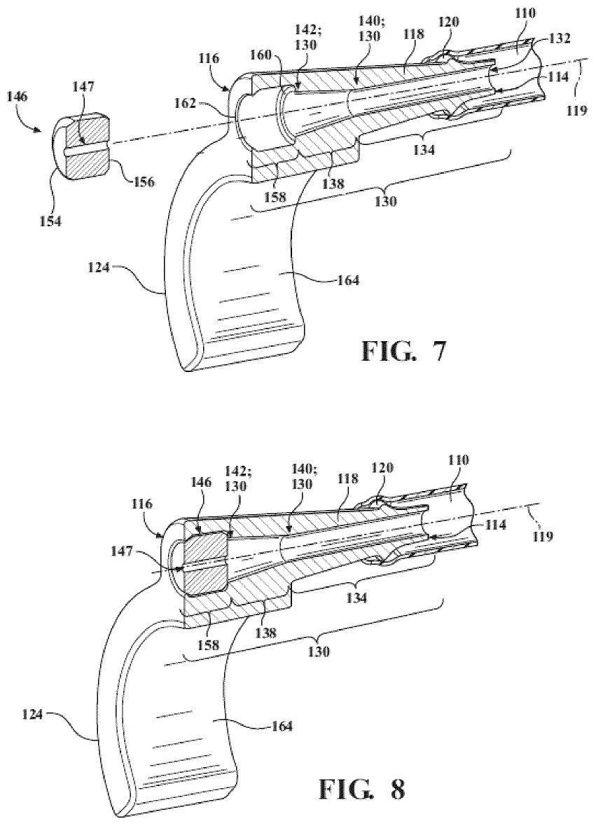

In some implementations, as best shown in FIGS. 7, 8, and 10-12, the connector 112 may be formed to include a cavity 158 opposite the lumen proximal end 132. In one interpretation, the cavity 158 may be considered part of the lumen 130. In another interpretation, the cavity 158 may be excluded from the lumen 130, but instead a feature formed at the distal lumen end. The cavity 158 is configured to receive a nozzle 146 such that the nozzle 146 is fluidly coupled with the lumen 130. The nozzle 146 includes a nozzle orifice 147. Unlike the previous implementations, here, the body 118 of the connector 112 is not formed to define an orifice 128. Instead, in this implementation, the body 118 defines the cavity 158 that is configured to receive the nozzle 146 (i.e., an insert) and the nozzle 146 defines the nozzle orifice 147. The nozzle 146 may be advantageous in situations wherein a higher resolution of manufacturing accuracy is required that current additive manufacturing techniques may not be able to provide.

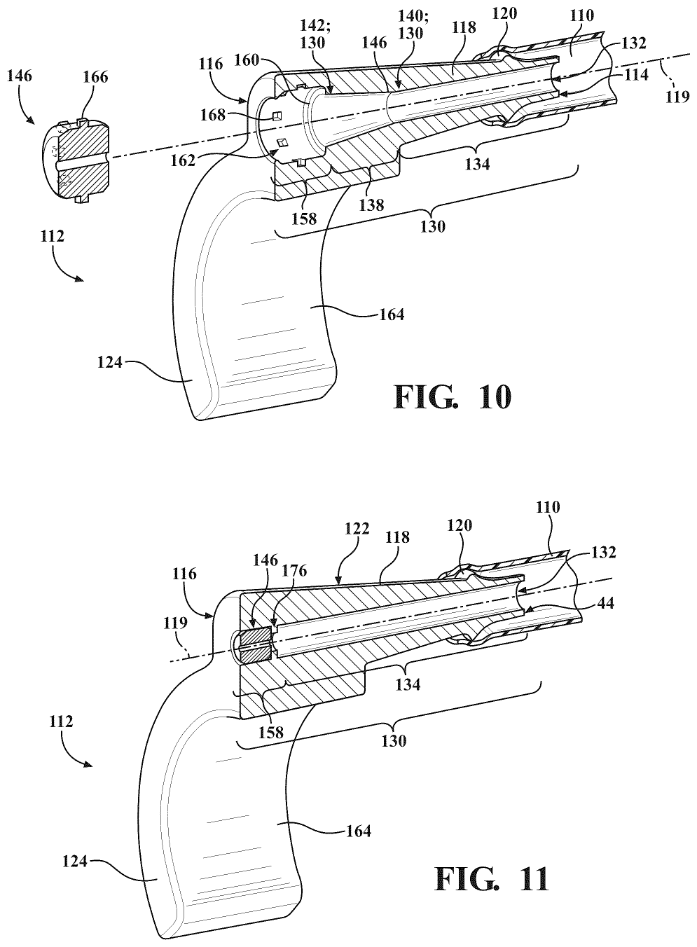

In the nozzle 146 configuration, the nozzle 146 is received by the cavity 158. The cavity 158 can include a cavity wall 161 that is perpendicular to a cavity base 160. As best shown in FIGS. 7 and 10, the cavity 158 also includes a cavity edge 162 located near the connector distal end 116. The second end 156 of the nozzle 146 abuts the cavity base 160. The nozzle 146 can be held by the cavity wall 161 and the nozzle 146 is captured by the cavity edge 162. The cavity edge 162 may have a concave geometry that reduces the cross-sectional area of the cavity 158 at the opening of the cavity 158. The reduced diameter can create an interference fit such that the nozzle 146 is pressed in the cavity 158 with some force to overcome the cavity edge 162. The cavity edge 162 presses on the nozzle 146 when the nozzle 146 is in the cavity 158 as shown in FIG. 8. The cavity 158 is shown with circular in cross-section but it should be appreciated that the cavity 158 may have any suitable geometry.

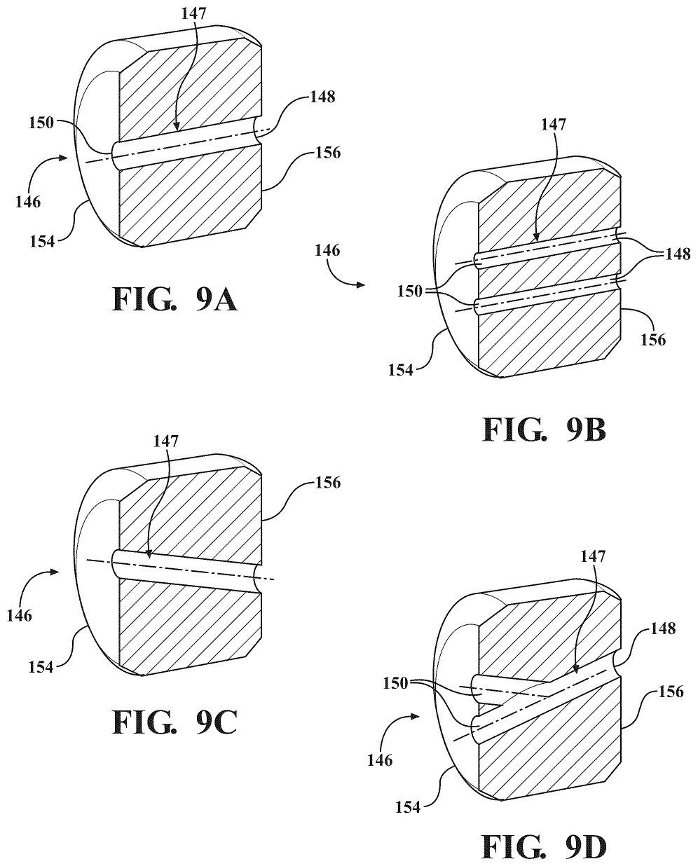

As best shown in FIGS. 9A-9D, the nozzle 146 defines at least one nozzle orifice 147 configured to receive fluid from the lumen 130 and expel fluid from the body 118 of the connector 112. The nozzle orifice 147 is different from the orifice 128 because the orifice 128 is integrally formed by the body 118 where the nozzle orifice 147 is formed by the nozzle 146 (e.g., a separate component from the body 118). Similar to the orifice 128, the nozzle orifice 147 has an inlet 148 that is configured to receive fluid from the transitional hollow 138 of the lumen 130. The nozzle orifice 147 also has an outlet 150 configured to expel fluid.

FIG. 9A-9D show multiple implementations of the nozzle orifice 147. The nozzle 146 includes at least one nozzle orifice 147 fully extending through the nozzle 146 and is in fluid communication with the lumen 130. The at least one nozzle orifice 147 is combined with the lumen 130 to provide the fluid path. In one interpretation, the at least one nozzle orifice 147 is a feature separate from the lumen 130. In another interpretation, the at least one nozzle orifice 147 may be considered part of the lumen 130. The nozzle orifice 147 has a cross-sectional area that is less than a cross-sectional area of the shaft segment 134. The nozzle orifice 147 can be parallel with the axis 119 of the lumen 130 or differ such that the nozzle orifice 147 is on another axis. The nozzle orifice 147 can include intersecting nozzle orifices 147 within the nozzle 146 as shown in FIG. 9D. The nozzle 146 may also include the transitional hollow 138 in fluid communication with the nozzle orifice 147 within the nozzle 146. The advantage of multiple nozzle orifices 147 is providing the ability to the user to select the particular nozzle orifice 147 based on the particular circumstances, such as the type of operation. The examples of the nozzle orifices 147 shown and described herein can equally be utilized for the orifices 128 formed by the body 118.

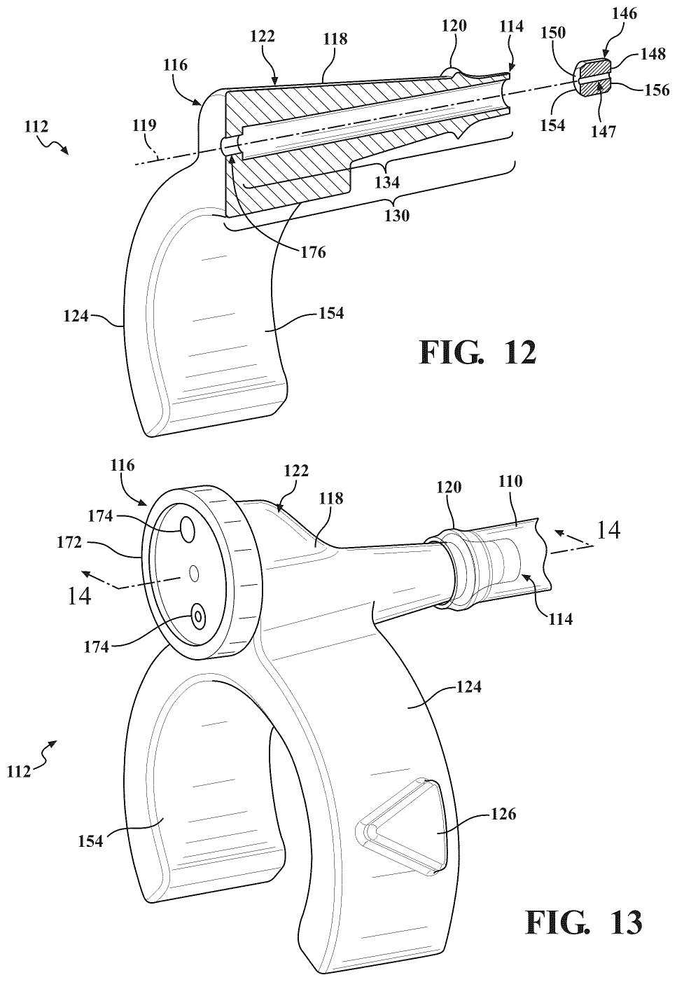

Referring to FIGS. 7 and 8, implementations of the connector 112 that include the nozzle 146 also include the transitional hollow 138 to connect the nozzle 146 with the lumen 130. As best shown in FIGS. 7, 8, and 10-12, in some implementations, the body 118 also defines a stop 176. The stop 176 can be adjacent to the transitional hollow 138. As best shown in FIGS. 10 and 11, the stop 176 is formed within the lumen 130, by the body 118 of the connector 112. The stop 176 of implementation shown in FIG. 11, also includes the cavity 158. The cavity 158 includes the cavity base 160, the cavity 158 and the cavity edge 162. The cavity 158 and nozzle 146 share the same diameter of the shaft segment 134. This implementation also shows the cavity 158 and nozzle 146 as smaller in diameter than previously shown. The stop 176 acts as a barrier for the nozzle 146 to set the depth within the lumen 130, The stop 176 also prevents the nozzle 146 from leaving the lumen 130 when in use. In FIG. 12, the stop 176 is formed at the lumen distal end. The nozzle 146 can be received by using a tool to press the nozzle 146 in the lumen 130 until the nozzle 146 abuts the stop 176.

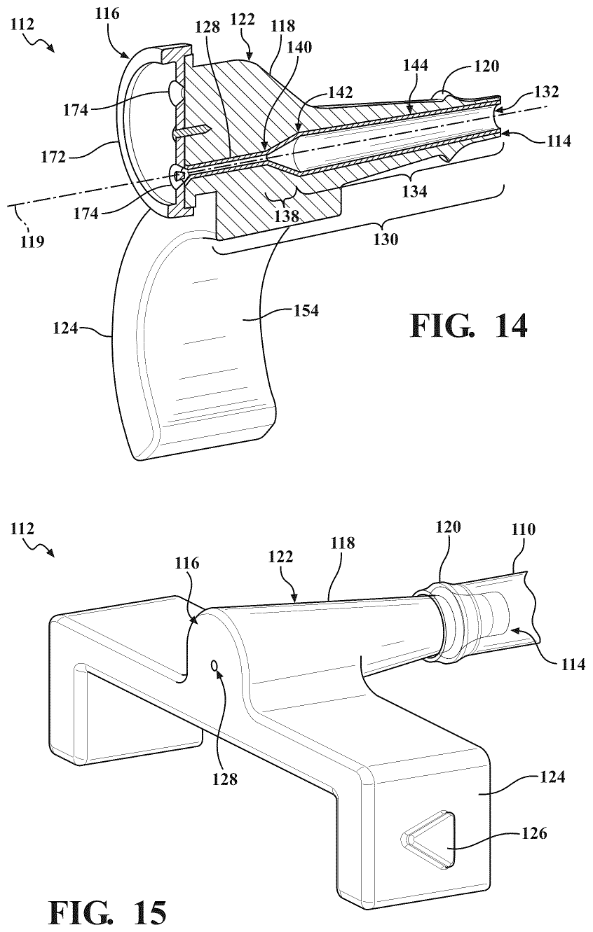

In some implementations, as shown in FIG. 13 and FIG. 14, the connector 112 includes a spray pattern adjuster 172 adjacent to the at least one orifice 128, 147, and wherein the spray pattern adjuster 172 is configured to adjust a spray pattern of the fluid that is expelled from the connector 112. In one example, the spray pattern adjuster 172 is configured to rotate to adjust a spray pattern of the fluid. The spray pattern adjuster 172 includes openings 174. The openings 174 control the flow of fluid from the at least one orifice 128, 147. The openings 174 can vary in size from one another. The spray pattern adjuster 172 provides the ability to quickly alter the spray pattern of fluid by enabling a user to select one of the openings 174. Changing the opening 174 to the orifice 128 changes the spray pattern which is used to control the amount of fluid that is dispensed. Various sized openings 174 are contemplated, such as a full circle opening, a line shaped opening, or the like. Further, an advantage of the openings 174 is having the ability to change the spray pattern without changing the nozzle 146. In other examples, the spray pattern adjuster 172 can change the opening 174 by pressing a button located on the connector 112.

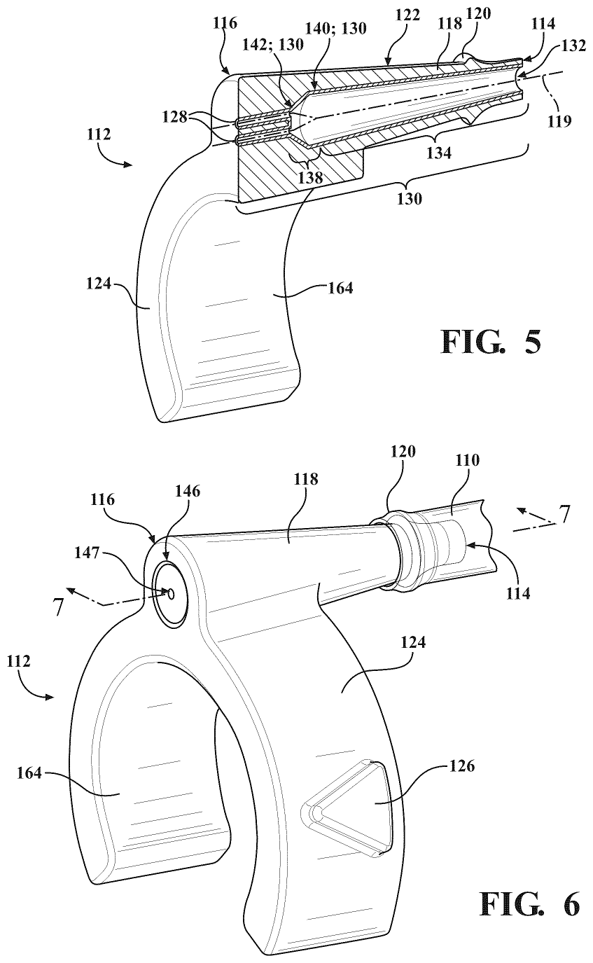

As best shown in FIG. 5, in some implementations, the body 118 of the lumen 130 is formed to include at least one orifice 128. The connector 112 may include more than one orifice 128 even when the connector 112 does not include the nozzle 146. The orifices 128 can vary in size and geometry.

In some implementations, as best shown in FIG. 10, the nozzle 146 and/or the cavity 158 define an anti-rotational mechanism configured to prevent the nozzle 146 from rotating within the cavity 158. For example, the body 118 may define a projection 168 that extends into the cavity 158 to capture the nozzle 146 as shown in FIG. 10. The nozzle 146 may define a recession 166 configured to mate with the projection 168 of the body 118 to capture the nozzle 146 within the cavity 158. The projection 168 and recession 166 may be swapped, such that the projection 168 extends from the nozzle 146 and the recession 166 is formed in the cavity 158. The recession 166 and projection 168 may be formed of any suitable shape. This anti-rotational design may be advantageous in situations where the nozzle 146 or its orifice(s) should be oriented in a specific manner, e.g., due to spray angle, or the like.

The nozzle 146 may comprise a metal or a metal alloy. Alternatively, the nozzle 146 may also comprise a resin based material or polymeric material. When the nozzle 146 may be formed from additive manufacturing.

Any of the described orifices 128, 147 can be designed as follows: with any type of angles of extension relative to the body axis 119 (e.g., transverse or parallel to the axis 119): with any type of orifice geometry that can implement different spray angles, spray directions, or spray ranges, or spray patterns: with any type of cross-sectional areas (e.g., consistent or varying);

any type of orifice length, or width; with any type of orifice path (e.g., straight path, curved path, curvilinear path); by converging or combining orifices; by diverging or splitting orifices; by parallel orifices; by colinear orifices; by concentric orifices; or any combination of any of the above features. The configuration of the orifices 128, 147 can be customized and specifically manufactured (additively) to adapt to a specific surgical procedure or indication, workflow, or surgeon preference requiring the fluid be dispersed in a particular way.

Moreover, the connector 112 can enable one nozzle 146 to be swapped for another nozzle 146 having a different configuration. This exchanging of nozzles 146 can occur anytime, e.g., during a procedure or between steps the procedure.

A method of manufacturing the connector 112 for attaching the fluid source 108 to the surgical tool 102 is also provided. The method includes forming the connector 112 by additive manufacturing. The connector 112 includes forming, by additive manufacturing, the attachment portion 124 extending from the body 118 transverse to the axis 119. Forming the connector 112 by additive manufacturing can include integrally forming the orifice 128 into the body 118.

When the nozzle 146 comprises a metal or a metal alloy, the method of forming the body 118 causes the body 118 to be in a semi-malleable state. The method may include inserting the nozzle 146 into the cavity 158 when the body 118 is in the semi-malleable state. In some implementations, joining the body 118 and the nozzle 146 is best accomplished when the body 118 is malleable. The method may include curing the nozzle 146 to the body 118. However, other processes of combining the body 118 and the nozzle 146 are also contemplated. For example, the body 118 and the nozzle 146 can be combined through ultrasonic welding or an adhesive.

Several implementations have been discussed in the foregoing description. However, the implementations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A connector for attaching a fluid source to a surgical tool, the connector comprising:
a body extending along an axis and defining a lumen within the body, wherein the lumen has a lumen proximal end configured to receive fluid from the fluid source, and wherein the lumen is configured to transport the fluid through the body; and
an attachment portion extending from the body transverse to the axis and configured to releasably attach the body to an exterior surface of the surgical tool; and
wherein the body and the attachment portion are integrally formed by additive manufacturing.

2. The connector of claim 1, wherein the body integrally defines at least one orifice through which the fluid is to be expelled, and wherein the at least one orifice has a cross-sectional area less than a cross-sectional area of the lumen at the lumen proximal end.

3. The connector of claim 2, wherein the lumen has a hollow shaft segment extending from the lumen proximal end towards the at least one orifice, wherein a cross-sectional area of the hollow shaft segment is uniform.

4. The connector of claim 3, wherein the lumen has a transitional hollow disposed between the hollow shaft segment and the at least one orifice, and wherein the transitional hollow has a cross-sectional area that decreases as the transitional hollow extends from the hollow shaft segment towards the at least one orifice.

5. The connector of claim 4, wherein the transitional hollow has a conical or funnel configuration.

6. The connector of claim 2, wherein the lumen and the at least one orifice extend along the axis of the body.

7. The connector of claim 2, wherein the at least one orifice extends along an axis that is transverse to the axis of the body.

8. The connector of claim 2, wherein the at least one orifice is further defined as a first orifice and a second orifice.

9. The connector of claim 8, wherein the first orifice and the second orifice differ from one another with respect to one or more of: cross-sectional area, angle of extension relative to the axis of the body, spray pattern, and/or spray angle.

10. The connector of claim 1, wherein a projection is formed on the attachment portion to assist with finger gripping the body.

11. The connector of claim 1, wherein the attachment portion is further defined as a pair of opposing arms that are flexible to engage with the exterior surface of the surgical tool.

12. The connector of claim 11, wherein each of the opposing arms is arc-shaped and configured to at least partially wrap around a cylindrical shaft of the surgical tool.

13. The connector of claim 1, wherein:
the body comprises a retention feature that annularly extends about an exterior surface of the body near the lumen proximal end;
the retention feature is configured to receive a tube that is coupled to the fluid source; and
the retention feature is configured to be disposed within the tube and retain the tube to the body with an interference fit.

14. The connector of claim 13, wherein the body has an arm portion defined between the attachment portion and the retention feature, and wherein the arm portion has a diameter that gradually decreases in size as the arm portion extends from the attachment portion to the retention feature.

15. The connector of claim 1, wherein the body is formed of a resin-based material or polymeric material.

16. The connector of claim 1, wherein the connector is a single-use device that is disposable.

17. The connector of claim 1, wherein an indicium is formed on the body by additive manufacturing, wherein the indicium conveys information.

18. A method of manufacturing a connector for attaching a fluid source to a surgical tool, the method comprising:
forming, by additive manufacturing, a body extending along an axis and defining a lumen within the body, and wherein the lumen is formed with a lumen proximal end configured to receive fluid from the fluid source, and wherein the lumen is formed to transport the fluid through the body; and
forming, by additive manufacturing, an attachment portion extending from the body transverse to the axis and wherein the attachment portion is formed to releasably attach the body to an exterior surface of the surgical tool.

19. The method of claim 18, comprising forming, by additive manufacturing, the body to integrally define at least one orifice through which the fluid is to be expelled, and wherein the least one orifice is formed with a cross-sectional area less than a cross-sectional area of the lumen at the lumen proximal end.

20. The method of claim 18, comprising forming the body and the attachment portion with a resin-based material and by using digital light processing (DLP).
